(19) United States
(12) Patent Application Publication
(10) Pub. No.: US 2004/0127747 A1
(43) Pub. Date: Jul. 1, 2004

(54) Synthesis of mono-N-substituted functionalized anilines

(75) Inventors: Maurizio Selva, Venezia (IT); Pietro Tundo, Venezia (IT)

Correspondence Address: MODIANO & ASSOCIATI, Via Meravigli, 16, MILANO, 20123, IT

(73) Assignee: CONSORZIO INTERUNIVERSITARIO NAZIONALE "LA CHIMICA PER L'AMBIENTE"

(21) Appl. No.: 10/734,208

(22) Filed: Dec. 15, 2003

(30) Foreign Application Priority Data
Dec. 18, 2002 (IT) PD2002A000325

(51) Int. Cl. C07C 233/00

(52) U.S. Cl. 564/154

(57) ABSTRACT

The present invention relates to a process for direct and selective synthesis of mono-N-substituted functionalized anilines by using alkylating agents selected from the class of organic carbonates, preferably of the dialkyl, dibenzyl and diallyl types, in the presence of suitable catalysts that are chemically related to the class of aluminosilicates.

### BACKGROUND OF THE INVENTION

The present invention relates to the synthesis of anilines mono-N-substituted and variously functionalized on the aromatic core.

Preparation of secondary aromatic amines has a primary role in many synthesis processes.

In particular, compounds that are the result of the introduction of alkyl, alkenyl and benzyl radicals on the aniline nitrogen atom are known for their importance in the synthesis of pharmaceutical components and in the color industry.

These products are usually obtained by direct reaction of primary anilines with alkyl halides or dialkylsulfates and allyl and benzyl halides, which are well-known in the chemical field for this application.

However, there have been cases in which this process entailed considerable difficulties.

In particular, reference is made here to the case in which the initial anilines were already functionalized (for example directly on the benzene core) with chemical groups such as hydroxyl, carboxyl and carboxyamine, which are potentially able to interfere due to their reactivities.

For example, direct alkylation of molecules such as aminophenols, aminobenzyl alcohols, aminobenzoic acids and aminobenzamides led to the unintended production of high percentages of N,N-dialkylated product (see in this regard i) Gibson, M. S.; The chemistry of the amino group 1968, Chapter 2, pages 45-62; ii) WO 9112000, and iii) Kalgutkar et al. J. Med. Chem. 1998, 41, 4800), and even to nonselective alkylation of other potentially interfering atoms.

In this regard, an alkylation that affected the molecules cited above, characterized by the presence of nucleophilic oxygen, would have led to N,O-dialkylated byproducts in final percentages that differed according to the reaction conditions used. (Yun, H. S. Chem. Abstr. 1974, 83, 42952).

In addition to reducing the overall yield of the intended product, this requires long and expensive purification procedures.

In order to minimize the incidence of these parasitic reactions, alternative synthetic approaches have been found which are based, for example, on the reductive amination of anilines by using formaldehyde or methyl esters (Krishnamurthy, S. Tetrahedron Letts. 1982, 3315), on the hydrolysis of benzoxazol derivatives (Sakurai, T.; Yamada, S.; Inoue, H. Bull. Chem. Soc. Jpn. 1986, 59, 2666), or on methods for selective O-dealkylation (Guo, Z.; Ramirez, J.; Li, J.; Wang, P. G. J. Am. Chem. Soc. 1998, 120, 3726).

These indirect methods usually entail lower yields than equivalent direct processes and, most of all, entail reaction conditions (particularly temperature and pressure) that are often drastic and incompatible with any labile functional groups.

Finally, despite the efforts made also in terms of obtaining the raw materials (which is not always easy), the problem of N,N-dialkylation still has not been solved (see in this regard EP-A-0855386, JP 52071424 and JP 2000095740).

Secondly, one should also remember that over the last decade the problem of possible environmental impact has become a decisive factor in assessing the applicability of new industrial processes and for any change to existing processes.

In this regard, the alkylating agents conventionally used (such as the already-mentioned alkyl halides and dialkylsulfates, and benzyl and allyl halides) are unfortunately known also for their considerable toxicity and for the particular conditions required in order to use them.

### SUMMARY OF THE INVENTION

The aim of the present invention is to provide a general process that allows to prepare the corresponding secondary aromatic amine by selective introduction of an alkyl, allyl or benzyl group at the nitrogen atom of a primary aniline functionalized at the aromatic ring.

Within this aim, an object of the present invention is to provide a direct synthesis process that leads to the mono-N-substitution of functionalized anilines, avoiding products of N,N-disubstitution (dialkyl, diallyl, or dibenzyl derivatives).

Another object of the present invention is to provide a direct and chemoselective process for mono-N-substitution at the nitrogen atom of functionalized anilines with groups that are potentially capable of interfering with said process.

A further object of the present invention is to provide a direct and selective synthesis method for mono-N-substitution at the nitrogen atom of functionalized anilines that is environmentally compatible as a whole.

This aim and these and other objects that will become better apparent hereinafter are achieved by a process for direct synthesis of mono-N-substituted anilines having the general formula (I)

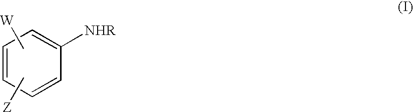

wherein

R indicates a linear or branched saturated carbon chain, preferably comprising 1 to 7 carbon atoms, an unsaturated carbon chain with the double carbon-carbon bond also in the allyl position (2,3) with respect to the nitrogen atom of the amines (I), comprising 3 to 7 carbon atoms, or a benzyl group or a benzyl group substituted at the aromatic ring with methyl and ethyl radicals;

W is selected from the group consisting of —H, —OH, —CH2OH, —COOH and —CONH2 and can be ortho, meta or para with respect to the carbon atom to which the nitrogen atom is attached;

Z is selected from the group consisting of —H, -halogen, -alkyl, -alkoxy, —NO2 and —CN; provided that W and Z are not simultaneously H,

said process comprising the step of reacting, in the presence of a solvent, a compound having the general formula (II):

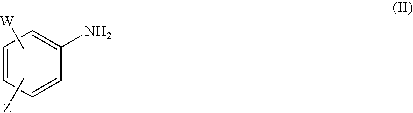

wherein W and Z are as defined above,

with an organic carbonate selected from the group consisting of compounds having the general formula (III)

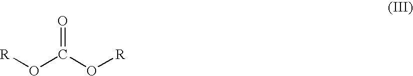

wherein R is as defined above,

and compounds having the general formula (IV)

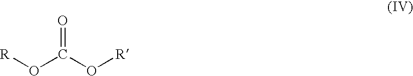

wherein R′ is selected from the group consisting of CH3(OCH2CH2)n- with n ≧2 and linear or branched alkyl radicals (where said radicals have in particular at least three carbon atoms), in the presence of a faujasite selected from the group consisting of X-faujasite exchanged with alkaline metals and Y-faujasite exchanged with alkaline metals.

Preferred examples of R groups according to the present invention are methyl, ethyl, allyl and benzyl.

Even more preferably, R is methyl and ethyl.

The term “alkyl” used in the context of the present invention designates a linear or branched saturated chain comprising 1 to 7 carbon atoms.

The term “alkoxy” used in the context of the present invention designates a linear or branched saturated chain joined to the aniline core by means of an oxygen bridge.

Preferably, the alkoxy radical comprises 1 to 5 carbon atoms. Preferred examples are methoxy, ethoxy, and propoxy.

According to the present invention, the reaction being considered is performed by placing in contact the aniline substrate, the organic carbonate and the faujasite.

The compounds (II) were made to react with the organic carbonate in the presence of faujasite at a temperature comprised between 70° C. and 190° C., preferably comprised between 90° C. and 150° C. and even more preferably comprised between 70° C. and 90° C. if dimethyl carbonate is used, between 70° C. and 130° C. if diethyl carbonate, diallyl carbonate and dibenzyl carbonate are used, and above 130° C. if the asymmetric carbonates having the formula (IV) are used.

Moreover, the process provided here allows to achieve the intended aim and objects even at atmospheric pressure, but this does not forbid the possibility to perform the alkylation reaction in an autoclave if required by the temperatures used.

In the specific case of methylation and ethylation reactions, if temperatures higher than the boiling points of the alkylating agents (DMC: 90° C.; DEC: 126° C.) are required, the reaction can still be performed at atmospheric pressure by using asymmetric carbonates having the general formula (IV):

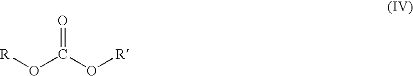

wherein R′ is an alkyl chain having 3 or more carbon atoms or an oxymethylene chain such as CH3(OCH2CH2)n- with n ≧2.

Oxymethylene derivatives are preferable because they are easier to synthesize and because of their low vapor pressure, which allows to use them over a wider temperature range.

In particular, preferred examples of carbonates having the formula (IV) are 2-(2-methoxyethoxy)ethyl-methylcarbonate (having the formula CH3(OCH2CH2)2OCO2Me) and 2-(2-methoxyethoxy)ethyl-ethylcarbonate (having the formula CH3(OCH2CH2)2OCO2Et).

The temperature preferably used for the carbonates having the formula (IV) is comprised between 130° and 190° C.

Regardless of the temperature and pressure conditions used, it has been found that the reaction is usually completed after a time ranging from 3 to 30 hours.

If the use as substrates of aniline derivatives that can be degraded easily as a consequence of hydrolytic or oxidative phenomena is desired, a modified atmosphere, for example by using inert gases such as nitrogen and argon is advantageously used in the present invention.

Generally, the organic carbonates (III) or (IV) used, if added in excess with respect to the organic substrate (II), are themselves perfectly capable of also acting as solvents.

Therefore, it has been found that the molar ratio of use of said alkylating agents with respect to the amount of aniline substrate can vary effectively between 10:1 and 50:1 and preferably between 25:1 and 35:1.

However, when the substrate has an excessively low coefficient of solubility in these solvents, it is possible to use co-solvents, among which mention is made here of dimethyl ethers derived from glycols and more particularly 1,2-dimethoxyethane (DME) and triglyme (triethylene glycol dimethyl ether).

These optional substances are used in a mixture with the carbonates (III) or (IV) and in ratios carbonate:co-solvent between 1:1 and 1:5 (v/v).

As noted above, the present invention provides a process in which the reaction conditions, particularly as regards the temperature, are particularly mild, and this aspect is a considerable advantage.

The achievement of high yields and excellent chemoselectivity even in these mild conditions has been achieved here mainly by way of the combined use of the carbonates cited above and of faujasites.

This last class of compounds indicates solids of the aluminosilicate type, which due to their structure can contain various types of ions of alkaline metals, alkaline-earth metals and/or transition metals.

Preferably, according to the present invention X-faujasites and Y-faujasites, both exchanged with alkaline metals, and even more preferably Y-faujasites exchanged with sodium, are used as catalysts.

As regards the quantities in which they are used, it has been found that it is possible to vary the percentages considerably while keeping, however, an interval of weight ratio: Y-faujasite/aniline substrate between 1:10 and 3:1 and more preferably between 1:1.5 and 1:1.

### DESCRIPTION OF THE PREFERRED EMBODIMENTS

Further characteristics and advantages of the process according to the present invention will become better apparent from the following detailed description of some preferred but not exclusive embodiments thereof.

### EXAMPLES

In the performed experimental tests reported here, the quantity of organic substrate (functionalized aniline) was varied over a range comprised between 0.5 g and 10 g, preferably 1 g.

Examples 1 to 7 and 12 refer to reactions performed in conventional reaction flasks and at atmospheric pressure, while examples 8 to 11 relate to processes performed in an autoclave.

### Example 1

N-methyl-p-aminophenol.

The p-aminophenol (1 g) is dissolved in a mixture consisting of dimethyl carbonate (hereafter referenced as DMC) (10 ml) and DME (35 ml).

The NaY-faujasite (1 g) is added to the resulting mixture and heating to 90° C. is performed.

The reaction, which is monitored by gas chromatography optionally associated with mass spectrometry, is completed after 7 hours, providing a practically quantitative conversion.

At this point the solid catalyst is eliminated by filtration and the monomethylated product can thus be recovered.

Instrumental analysis performed after solvent evaporation yielded a selectivity value of 99% for the N-monomethylated product and a total yield of 91%.

### Example 2

N-methyl-o-aminophenol

The o-aminophenol (1 g) is dissolved in pure DMC (30 ml).

In this case, no co-solvent is added.

The NaY-faujasite (1 g) is added to the resulting mixture and heating to 90° C. is performed.

The reaction, which is monitored by means of gas chromatography optionally associated with mass spectrometry, is completed after 3 hours, providing a practically quantitative conversion.

At this point the solid catalyst is eliminated by filtration and the monomethylated product can thus be recovered.

Instrumental analysis performed after solvent evaporation reveals a total yield of 99% of N-monomethylated product.

### Example 2a

The method of example 2 is repeated, but in this case the quantity of NaY-faujasite is limited to 1/10 of the quantity of o-aminophenol (again 1 g).

The reaction is completed after 38 hours, giving a total yield of 93% of N-monomethylated product.

### Example 3

N-methyl-m-aminophenol

A mixture of m-aminophenol (1 g) in DMC (30 ml) is prepared according to the method described in example 1 without resorting to co-solvents.

In this case, the NaY-faujasite is added in a quantity that is equal in weight with respect to the m-aminophenol.

The reaction is performed for 7 hours.

The yield of N-methyl-m-aminophenol is 89%.

### Example 3a

The reaction of example 3 is reproduced, again starting from m-aminophenol (1 g), but in this case the quantity of NaY-faujasite is 1/10 with respect to the aromatic substrate.

The time required for the reaction to complete is 42 hours, but the final yield still remains high and is more precisely equal to 92%.

### Example 4

N-methyl-p-aminobenzyl alcohol

The p-aminobenzyl alcohol (1 g) is dissolved in pure DMC (30 ml).

The NaY-faujasite (1 g) is added to the resulting mixture and heating to 90° C. is performed.

The reaction, which is monitored by gas chromatography optionally associated with mass spectrometry, is completed after 8 hours.

The mono-N-methylation selectivity is 94%.

The solid catalyst (NaY-faujasite) is again eliminated by filtration.

The product is recovered after purification performed with flash chromatography (eluent: ethyl acetate/petroleum ether, 1:4 v/v).

The final yield on N-methyl-p-aminobenzyl alcohol is equal to 77%.

### Example 5

N-methyl-o-aminobenzyl alcohol

A mixture of o-aminobenzyl alcohol (1 g) in DMC (30 ml) is prepared according to the method described in example 1.

The two compounds are made to react in the presence of NaY-faujasite (1 g) for 12 hours in the conditions described in the preceding examples.

The yield on the isolated title product is 92%.

The mono-N-methylation selectivity is 99%.

### Example 6

N-methyl-p-aminobenzamide

According to the method described in example 1, the p-aminobenzamide (1 g) is made to react with the DMC (50 ml), which also acts as a solvent.

After 24 hours, the reaction is completed, with 93% mono-N-methylation selectivity.

The methylated product is recovered by flash column chromatography (eluent: ethyl acetate/petroleum ether, 1:4 v/v) and the final yield is 86%.

### Example 7

N-methyl-o-aminobenzamide

The protocol of example 6 is now repeated, except that the quantity of DMC is slightly reduced (30 ml).

The reaction is extended for 22 hours, leading to a yield after purification of 91%.

### Example 8

N-methyl-p-aminobenzoic acid

A solution of p-aminobenzoic acid (1 g) in DMC (30 ml) is prepared and the NaY-faujasite according to the protocol of example 1 is then added.

However, differently from the preceding examples, in this case the temperature to which the mixture is brought is 130° C. and therefore the reaction must be performed in an autoclave (it is noted in this regard that the boiling point of DMC is 90° C.).

After 9 hours, the raw reaction product is recovered and purified by flash column chromatography (eluent: ethyl acetate/petroleum ether, 1:4 v/v).

The yield of mono-N-methylation on the isolated product is 74%.

### Example 9

N-methyl-o-aminobenzoic acid

The protocol of example 8 is repeated, except that the temperature at which the reaction is performed is 150° C.

After 5 hours, the product is recovered, obtaining a yield equal to 83%.

### Example 10

Ethyl N-methyl-o-aminobenzoate

In the same manner as in example 8, the ethyl o-aminobenzoate (1 g) is made to react in an autoclave at 150° C. with the DMC (30 ml) and, obviously, in the presence of the solid catalyst.

After 8 hours, analysis of a sample by gas chromatography confirms that the only product of the reaction is ethyl N-methyl-o-aminobenzoate (65% yield calculated on the chromatogram), while there are no traces of the product derived from the possible transesterification of the ethyl with the methyl (which would have yielded methyl N-methyl-o-aminobenzoate).

### Example 11

Methyl N-ethyl-o-aminobenzoate

The alkylation reaction of the preceding example is reproduced here, but the DMC is replaced with diethyl carbonate (30 ml), while the substrate remains o-aminobenzoate (1 g).

After 8 hours, analysis of a sample by gas chromatography confirms that the only product of the reaction is methyl N-ethyl-o-aminobenzoate (23% yield at the time of analysis calculated on the chromatogram), while there are no traces of the product that derives from the possible transesterification of the ethyl with the methyl (which would have yielded ethyl N-ethyl-o-aminobenzoate).

### Example 12

N-methyl-o-aminobenzoic acid

The o-aminobenzoic acid (1 g) is dissolved in a mixture constituted by triethylene glycol dimethyl ether (triglyme, 15 ml) and 2-(2-methoxyethoxy)ethyl-methylcarbonate (CH3(OCH2CH2)2OCO2CH3, 7 ml).

The NaY-faujasite (1 g) is added to the mixture and heating to 150° C. is performed at atmospheric pressure, in the presence of inert gas.

After 12 hours, the reaction is complete: gaschromatographic analysis indicates the product of mono-N-methylation at 93%.

### Example 13

N-ethyl-o-aminobenzoic acid

The o-aminobenzoic acid (1 g) is dissolved in a mixture constituted by triethylene glycol dimethyl ether (triglyme, 15 ml) and 2-(2-methoxyethoxy)ethyl-ethylcarbonate [Me(OCH2CH2)2OCO2Et, 7 ml].

The NaY-faujasite (1 g) is added to the mixture and heating is performed to 150° C., at atmospheric pressure, in the presence of inert gas.

After 18 hours, conversion is 87%: gaschromatographic analysis indicates a mono-N-ethylation selectivity of 97%.

One considerably important aspect revealed by the cited examples (in particular by examples 1-8) is that the present invention allows to use organic carbonates while maintaining reaction temperatures that are distinctly lower than those cited in the background art (Trotta, F.; Tundo, P.; Moraglio, G.; J. Org. Chem. 1987, 52, 1300).

The advantages that arise from the adoption of low temperatures are many and are well-known to anyone working in this field.

Among them, mention is made in any case of the possibility to use substrates that are sensitive to heat degradation and most of all to use relatively light organic carbonates (with boiling points generally lower than 130° C.) as solvents without necessarily having to resort to autoclaves.

Shieh W.-C. et al. have reported (Shieh, W.-C.; Dell, S.; Repic, O. Org. Lett. 2001, 3, 4279 and J. Org. Chem. 2002, 67, 2188) that DMC can be used effectively as an alkylating agent at low temperatures only in sporadic cases and in any case either by adding a non-nucleophilic strong base (such as DBU) as a catalyst or by triggering the reaction with microwaves.

Chemical literature, further, reports that basic or acid catalysts can greatly modify the selectivity of alkylation reactions that use dialkyl carbonates.

The following example is provided as a confirmation of what is described in the background art.

### Example 14 (for comparison)

p-Aminophenol (1 g) is dissolved in a mixture constituted by dimethyl carbonate (10 ml) and dimethyl formamide (15 ml).

Potassium carbonate (2 equivalents for each equivalent of p-aminophenol) is added to the resulting solution as a catalyst and heating to 125° C. is performed.

After 5 hours, instrumental analysis of a sample of the mixture showed that conversion was 77% but showed also that the reaction had not yielded a single product.

In particular, the N,O-dimethylate product (22%), the O-monomethylate product (23%), the N,N,O-trimethylate product (18%), further O-methylated urethane (p-CH3OC6H4NHCO2CH3, 6%), and N,O-dimethylated urethane (8%) were identified.

From what is reported in the literature, it is reasonable to believe that the same lack of selectivity shown in example 14 can also be found if the initial substrate is constituted by aminobenzamides or aminobenzoic acids.

Therefore, it is now evident that the combination according to the present invention between organic carbonates [(III) or (IV)] and faujasites as catalysts allows to work at low temperatures while maintaining, and often even improving, the yield and most of all the selectivity of known equivalent processes.

The disclosures in Italian Patent Application No. PD2002A000325 from which this application claims priority are incorporated herein by reference.

What is claimed is:

1. A process for the direct synthesis of mono-N-substituted anilines having the general formula (I) 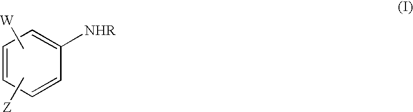
wherein
R indicates a linear or branched saturated carbon chain, preferably comprising 1 to 7 carbon atoms, an unsaturated carbon chain with the double carbon-carbon link also in the allyl position (2,3) with respect to the nitrogen atom of the amines (I), comprising 3 to 7 carbon atoms, or a benzyl group or a benzyl group substituted at the aromatic ring with methyl and ethyl radicals;
W is selected from the group consisting of —H, —OH, —CH2OH, —COOH and —CONH2 and can be ortho, meta or para with respect to the carbon atom to which the nitrogen atom is attached;
Z is selected from the group consisting of —H, -halogen, -alkyl, -alkoxy, —NO2 and —CN; provided that W and Z are not simultaneously H,
said process comprising the step of reacting, in the presence of a solvent, a compound having the general formula (II): 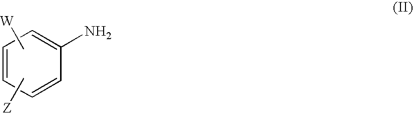
wherein W and Z are as defined above,
with an organic carbonate selected from the group consisting of compounds having the general formula (III) 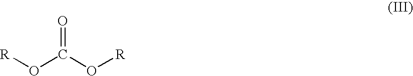
wherein R is as defined above,
and compounds of general formula (IV) 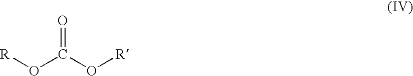
wherein R′ is selected from the group consisting of CH3(OCH2CH2)n- with n≧2 and branched or linear alkyl radicals that have at least three carbon atoms;
in the presence of a faujasite selected from the group comprising X-faujasite exchanged with alkaline metals and Y-faujasite exchanged with alkaline metals.

2. The process according to claim 1, wherein the organic carbonate has the general formula (III).

3. The process according to claim 1, wherein the organic carbonate has the general formula (IV).

4. The process according to claim 1, wherein the radical R is selected from the group consisting of methyl, ethyl, allyl and benzyl.

5. The process according to claim 1, wherein said faujasite is present in a ratio between 1:10 and 3:1 with respect to the compound having the formula (I).

6. The process according to claim 5, wherein said faujasite is present in a ratio between 1:1.5 and 1:1 with respect to the compound having the formula (I).

7. The process according to claim 1, wherein said faujasite is Y-faujasite exchanged with sodium.

8. The process according to claim 1, wherein said organic carbonate is dimethyl carbonate.

9. The process according to claim 1, wherein said organic carbonate is diethyl carbonate.

10. The process according to claim 1, wherein said organic carbonate is diallyl carbonate.

11. The process according to claim 1, wherein said organic carbonate is dibenzyl carbonate.

12. The process according to claim 1, wherein said organic carbonate is 2-(2-methoxyethoxy)ethyl-methylcarbonate.

13. The process according to claim 1, wherein said organic carbonate is 2-(2-methoxyethoxy)ethyl-ethylcarbonate.

14. The process according to claim 1, wherein the organic carbonate is present in a ratio between 10:1 and 50:1 with respect to the compound having the formula (I).

15. The process according to claim 1, wherein said step is performed at a temperature between 70° C. and 190° C.

16. The process according to claim 15, wherein said temperature is between 90° C. and 150° C.

17. The process according to claim 8, wherein said step is performed at a temperature between 70° C. and 90° C.

18. The process according to claim 9, wherein said step is performed at a temperature between 70° C. and 130° C.

19. The process according to claim 10, wherein said step is performed at a temperature between 130° C. and 190° C.

20. The process according to claim 11, wherein said step is performed at a temperature between 130° C. and 190° C.

21. The process according to claim 1, wherein said step is performed at atmospheric pressure.

22. The process according to claim 1, wherein said step is performed in an autoclave.

23. The process according to claim 1, wherein said step is performed in atmosphere that is modified by adding an inert gas selected from the group consisting of nitrogen and argon.

24. The process according to claim 1, wherein said solvent is selected from the group consisting of:
said organic carbonate,
a co-solvent, and
mixtures thereof.

25. The process according to claim 24, wherein said co-solvent is selected from the group consisting of 1,2-dimethoxyethane, triethylene glycol dimethyl ether, and mixtures thereof.

26. The process according to claim 24, wherein said solvent is a mixture of said organic carbonate and of a co-solvent and said co-solvent is preferably present in a ratio comprised between 1:1 and 5:1 with respect to the organic carbonate.

27. The use of faujasites exchanged with alkaline metals to catalyze reactions for mono-N-substitution of functionalized anilines.

28. The use according to claim 27, wherein said faujasite is NaY-faujasite.
